(19) United States
(12) Patent Application Publication
(10) Pub. No.: US 2023/0416189 A1
(43) Pub. Date: Dec. 28, 2023

(54) NEW SYNTHESIS OF L-PHENYLALANINE BUTYRAMIDE

(71) Applicant: DSM IP ASSETS B.V., Heerlen (NL)

(72) Inventors: Werner BONRATH, Kaiseraugst (CH); Alexander GAA, Kaiseraugst (CH); Ralph HAERTER, Kaiseraugst (CH)

(21) Appl. No.: 18/253,178

(22) PCT Filed: Nov. 9, 2021
(86) PCT No.: PCT/EP2021/081029

§ 371 Date: May 16, 2023

(30) Foreign Application Priority Data
Nov. 18, 2020 (EP) 20208401.8

(51) Int. Cl. C07C 231/02

(52) U.S. Cl. CPC C07C 231/02

(57) ABSTRACT

The present invention relates to a process for producing L-phenylalanine butyramide.

The present invention relates to a process for producing L-phenylalanine butyramide.

L-Phenylalanine butyramide is an important derivative of the butyric acid (BUT).

It was shown that phenylalanine-butyramide protects against experimental doxorubicin cardiotoxicity. Such protection is accompanied by reduction in oxidative stress and amelioration of mitochondrial function.

EP2268605 discloses a method to produce L-phenylalanine butyramide starting from phenylalanine carboxamide in chloroform as solvent. The yield is around 50 to 60%.

Because L-phenylalanine butyramide is an important compound there is always a need for improved ways to produce it.

Surprisingly, a new process was found, which is free of chlorinated solvents and wherein the reaction results in excellent yield.

L-Phenylalanine butyramide is the following compound of formula (I)

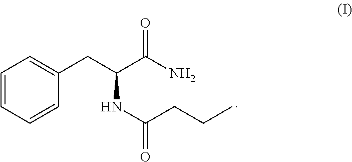 [SMILES: CCCC(=O)N[C@@H](Cc1ccccc1)C(N)=O]

The new and improved synthesis of L-phenylalanine amide allows to obtain L-phenylalanine butyramide in an excellent yield without the use of any chlorinated solvents.

Therefore, the present invention relates to process (P) to produce L-phenylalanine butyramide, which is the compound of formula (I)

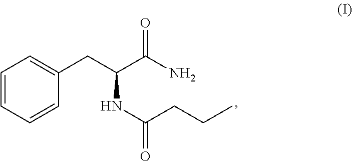 [SMILES: CCCC(=O)N[C@@H](Cc1ccccc1)C(N)=O]

wherein a first step (step (i)) the compound of formula (II)

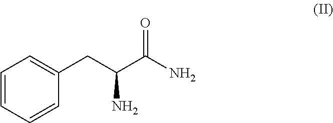 [SMILES: NC(=O)[C@@H](N)Cc1ccccc1]

is reacted with a compound of formula (III)

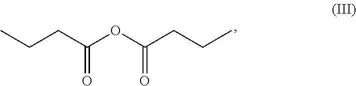

and in a second step (step (ii)) water is added to the reaction mixture of step (i).

The process according to the present invention is usually carried out as following:

In a first step (step (i)) the compound of formula (II) is reacted with the compound of formula (III) at a temperature of 20° C. to 35° C.

Afterwards in a second step (step (ii)) water is added the reaction mixture of the first step. Finally, the product (compound of formula (I)) is removed from the reaction mixture and purified.

The process according to the present invention is carried out without any chlorinated solvent.

The compound of formula (III), which is butyric anhydride, serves also as the solvent and therefore is added in molar excess (in regard to the compound of formula (II)) to the reaction mixture.

The compound of formula (III) can be used in any molar excess. Usually, the molar ratio of the compound of formula (III) to the compound of formula (II) in step (i) is at least 2:1. The upper limit is not essential for the invention. Usually it is up to 100:1. A preferred molar ratio of the compound of formula (III) to the compound of formula (II) is usually from 5:1 to 50:1.

It is possible to use an additional non-chlorinated inert solvent (or a mixture of non-chlorinated inert solvents).

As stated above the process according to the present invention is carried out in the absence of any chlorinated solvent.

Preferably, the reaction of the present invention is carried without any additional solvent (next to the compound of formula (II) and water).

Therefore, the present invention relates to process (P1), which is process (P), wherein step (i) the molar ratio of the compound of formula (III) to the compound of formula (II) is at least 2:1.

Therefore, the present invention relates to process (P1′), which is process (P), wherein step (i) the molar ratio of the compound of formula (III) to the compound of formula (II) is from 2:1 to 100:1.

Therefore, the present invention relates to process (P1″), which is process (P), wherein step (i) the molar ratio of the compound of formula (III) to the compound of formula (II) is from 5:1 to 50:1.

Therefore, the present invention relates to process (P2), which is process (P), (P1), (P1′) or (P1″), wherein the process is carried without any chlorinated solvent.

Therefore, the present invention relates to process (P3), which is process (P), (P1), (P1′), (P1″) or (P2), wherein the process is carried without any additional solvent (next to the compound of formula (II) and water).

Usually step (i) is carried out at a temperature of 20° C. - 35° C. (preferably from 20° C. — 30° C.).

Therefore, the present invention relates to process (P4), which is process (P), (P1), (P1′), (P1″), (P2) or (P3), wherein step (i) is carried out at a temperature of 20° C. -35° C.

Therefore, the present invention relates to process (P4′), which is process (P), (P1), (P1′), (P1″), (P2) or (P3), wherein step (i) is carried out at a temperature of 20° C. -30° C.

Usually step (i) of the process according to the present invention is carried out at ambient pressure.

Therefore, the present invention relates to process (P5), which is process (P), (P1), (P1′), (P1″), (P2), (P3), (P4) or (P4′), wherein step (i) is carried out at ambient pressure.

Afterwards when the reaction of step (i) has been carried out, water is added to the reaction mixture (as obtained from step (i)).

Usually step (ii) of the process according to the present invention is carried out at ambient pressure.

Therefore, the present invention relates to process (P6), which is process (P), (P1), (P1′), (P1″), (P2), (P3), (P4), (P4′) or (P5), wherein step (ii) is carried out at ambient pressure.

The water, which is added in step (ii) is usually distilled water and it is cold (temperature of the water is below 20° C.). Usually, the temperature of the water to be added is 5-15° C.

In step (ii), the water is added usually in excess in view of the compound of formula (II). Usually, it is added in large excess and the amount of the added water is not essential or critical for the process according to the present invention.

Usually, the molar ratio of the added water in step (ii) to the compound of formula (II) is at least 5.1.

The upper limit is not essential for the invention. Usually it is up to 500:1. A preferred molar ratio of the compound of formula (III) to the compound of formula (II) is usually from 5:1 to 200:1.

Therefore, the present invention relates to process (P7), which is process (P), (P1), (P1′), (P1″), (P2), (P3), (P4), (P4′), (P5) or (P6), wherein the water to be added in step (ii) is distilled water.

Therefore, the present invention relates to process (P8), which is process (P), (P1), (P1′), (P1″), (P2), (P3), (P4), (P4′), (P5), (P6) or (P7), wherein the water to be added in step (ii) has a temperature of below 20° C.

Therefore, the present invention relates to process (P8′), which is process (P), (P1), (P1′), (P1″), (P2), (P3), (P4), (P4′), (P5), (P6) or (P7), wherein the water to be added in step (ii) has a temperature of from 5-15° C.

Therefore, the present invention relates to process (P9), which is process (P), (P1), (P1′), (P1″), (P2), (P3), (P4), (P4′), (P5), (P6), (P7), (P8) or (P8′), wherein the water is added in excess in view of the compound of formula (II).

Therefore, the present invention relates to process (P9′), which is process (P), (P1), (P1′), (P1″), (P2), (P3), (P4), (P4′), (P5), (P6), (P7), (P8) or (P8′), wherein the molar ratio of the added water in step (ii) to the compound of formula (II) is at least 5:1.

Therefore, the present invention relates to process (P9″), which is process (P), (P1), (P1′), (P1″), (P2), (P3), (P4), (P4′), (P5), (P6), (P7), (P8) or (P8′), wherein the molar ratio of the added water in step (ii) to the compound of formula (II) is 5:1 to 500:1.

Therefore, the present invention relates to process (P9″), which is process (P), (P1), (P1′), (P1″), (P2), (P3), (P4), (P4′), (P5), (P6), (P7), (P8) or (P8′), wherein the molar ratio of the added water in step (ii) to the compound of formula (II) is 5:1 to 200:1.

Afterwards the product (compound of formula (I)) is isolated from the reaction mixture (and optionally purified) by usual means.

L-Phenylalanine butyramide is obtained in excellent yields.

The following Example illustrates the invention further without limiting it. All percentages and parts, which are given, are related to the weight and the temperatures are given in ° C., when not otherwise stated.

### EXAMPLES

### Example 1

In a flask 10 ml (59 mmol) anhydrous butyric anhydride was placed. Under stirring at 25° C. 1g (6.09 mmol) of phenylalanine amide is added in portions during 10 min.

The mixture was stirred overnight (17 h) at 400 rpm at 25° C.

Then 10 ml of ice-cold distilled water is added, and the mixture stirred for 30 min on ice-cold water bath. Then 17 ml of ice-cold distilled water are added and the obtained solid is filtered.

The solid is washed 3 times with 10 ml of ice-cold water and the residue is re-dissolved in 67 ml of boiling water. After spontaneous cooling withe needle-shaped crystals are formed and filtered.

The compound of formula (I) was obtained in a yield of 82%.

1. Process to the compound of formula (I) 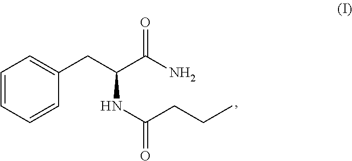 [SMILES: CCCC(=O)N[C@@H](Cc1ccccc1)C(N)=O]
wherein a first step (step (i)) the compound of formula (II) 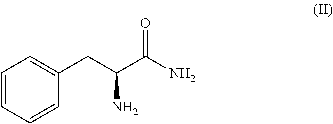 [SMILES: NC(=O)[C@@H](N)Cc1ccccc1]
is reacted with a compound of formula (III) 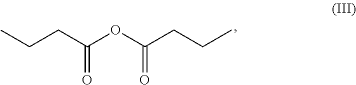
and in a second step (step (ii)) water is added to the reaction mixture of step (i).

2. Process according to claim 1, wherein step (i) the molar ratio of the compound of formula (III) to the compound of formula (II) is at least 2:1.

3. Process according to claim 1, wherein the molar ratio of the compound of formula (III) to the compound of formula (II) is from 2:1 to 100:1.

4. Process according to claim 1, wherein the process is carried without any chlorinated solvent.

5. Process according to claim 1, wherein step (i) is carried out at a temperature of 20° C.-35° C.

6. Process according to claim 1, wherein step (i) is carried out at ambient pressure.

7. Process according to claim 1, wherein step (ii) is carried out at ambient pressure.

8. Process according to claim 1, wherein the water to be added in step (ii) is distilled water.

9. Process according to claim 1, wherein the water to be added in step (ii) has a temperature of below 20° C.

10. Process according to claim 1, wherein the water is added in excess in view of the compound of formula (II).

11. Process according to claim 10, wherein the molar ratio of the added water in step (ii) to the compound of formula (II) is at least 5:1.
